(19) United States
(12) Patent Application Publication
(10) Pub. No.: US 2025/0021746 A1
(43) Pub. Date: Jan. 16, 2025

(54) DOCUMENT CREATION SUPPORT APPARATUS, DOCUMENT CREATION SUPPORT METHOD, AND DOCUMENT CREATION SUPPORT PROGRAM

(71) Applicant: FUJIFILM Corporation, Tokyo (JP)

(72) Inventors: Shotaro MISAWA, Tokyo (JP); Nobuya KITAMURA, Tokyo (JP); Yukie SUGIYAMA, Tokyo (JP); Hiroki SATO, Tokyo (JP); Hirokazu YARIMIZU, Tokyo (JP); Kohei ONODA, Tokyo (JP)

(73) Assignee: FUJIFILM Corporation, Tokyo (JP)

(21) Appl. No.: 18/748,064

(22) Filed: Jun. 19, 2024

(30) Foreign Application Priority Data
Jul. 11, 2023 (JP) 2023-114075

(51) Int. Cl. G06F 40/166; G06F 40/279

(52) U.S. Cl. CPC G06F 40/166; G06F 40/279

(57) ABSTRACT

A document creation support apparatus displays a subject document creation screen for creating a subject document and a reference document display screen for displaying a reference document to be used for creating the subject document, and in a case where a plurality of the reference documents are displayed on the reference document display screen, displays an overlapping part included in the reference document in a non-highlighted manner with respect to a non-overlapping part.

### CROSS-REFERENCE TO RELATED APPLICATIONS

This application claims priority from Japanese Patent Application No. 2023-114075, filed on Jul. 11, 2023, the entire disclosure of which is incorporated herein by reference.

### BACKGROUND

### 1. Technical Field

The present disclosure relates to a document creation support apparatus, a document creation support method, and a document creation support program.

### 2. Description of the Related Art

JP2010-224639A discloses a technology for extracting a description from a reference document that is created in advance and that is referred to at a time of creating a summary document based on a predetermined extraction condition, extracting a description from the summary document based on an extraction condition, and learning a condition to be added to the extraction condition based on a comparison between the description extracted from the reference document and the description extracted from the summary document. In this technology, a description to be used for creating a summary from an electronic document as a target for creating the summary is extracted based on the extraction condition and on the learned condition.

### SUMMARY

In a specific field, a subject document may be created using content of a plurality of reference documents while the content of the reference documents created daily is viewed after an elapse of a certain period. For example, in the medical field, the summary document is created as the subject document by summarizing the content of the plurality of reference documents while the plurality of reference documents such as daily charts are viewed at a specific time such as a time of hospital discharge. Meanwhile, in order to create the summary document, it is required to check information required for creating the subject document from a large amount of information included in the plurality of reference documents, and time and effort are required for creating the summary document. Thus, support for creating the summary document is required.

The present disclosure is conceived in view of the above circumstances, and an object of the present disclosure is to provide a document creation support apparatus, a document creation support method, and a document creation support program that can effectively support creation of a subject document using a plurality of reference documents.

A document creation support apparatus according to an aspect of the present disclosure is a document creation support apparatus comprising at least one processor, in which the processor is configured to display a subject document creation screen for creating a subject document and a reference document display screen for displaying a reference document to be used for creating the subject document, and in a case where a plurality of the reference documents are displayed on the reference document display screen, display an overlapping part included in the reference document in a non-highlighted manner with respect to a non-overlapping part.

In addition, a document creation support method according to another aspect of the present disclosure is a document creation support method comprising, via a processor included in a document creation support apparatus, displaying a subject document creation screen for creating a subject document and a reference document display screen for displaying a reference document to be used for creating the subject document, and displaying, in a case where a plurality of the reference documents are displayed on the reference document display screen, an overlapping part included in the reference document in a non-highlighted manner with respect to a non-overlapping part.

In addition, a document creation support program according to still another aspect of the present disclosure is a document creation support program for causing a processor included in a document creation support apparatus to execute a process comprising displaying a subject document creation screen for creating a subject document and a reference document display screen for displaying a reference document to be used for creating the subject document, and displaying, in a case where a plurality of the reference documents are displayed on the reference document display screen, an overlapping part included in the reference document in a non-highlighted manner with respect to a non-overlapping part.

According to the present disclosure, creation of a subject document using a plurality of reference documents can be effectively supported.

### BRIEF DESCRIPTION OF THE DRAWINGS

FIG. 1 is a block diagram illustrating an example of a configuration of an information processing system.

FIG. 2 is a block diagram illustrating an example of a hardware configuration of a user terminal.

FIG. 3 is a block diagram illustrating an example of a hardware configuration of an information processing apparatus.

FIG. 4 is a diagram illustrating an example of a display screen.

FIG. 5 is a block diagram illustrating an example of a functional configuration of the information processing apparatus.

FIG. 6 is a diagram for describing overlap determination processing for partial documents.

FIG. 7 is a block diagram illustrating an example of a functional configuration of the user terminal.

FIG. 8 is a diagram showing an example of a display screen.

FIG. 9 is a diagram illustrating an example of a reference document display screen.

FIG. 10 is a diagram illustrating an example of the reference document display screen.

FIG. 11 is a diagram illustrating an example of the reference document display screen.

FIG. 12 is a diagram for describing copy processing.

FIG. 13 is a diagram illustrating an example of the reference document display screen.

FIG. 14 is a flowchart illustrating an example of the overlap determination processing.

FIG. 15 is a flowchart illustrating an example of display control processing.

### DETAILED DESCRIPTION

Hereinafter, an embodiment of the disclosed technology will be described in detail with reference to the drawings.

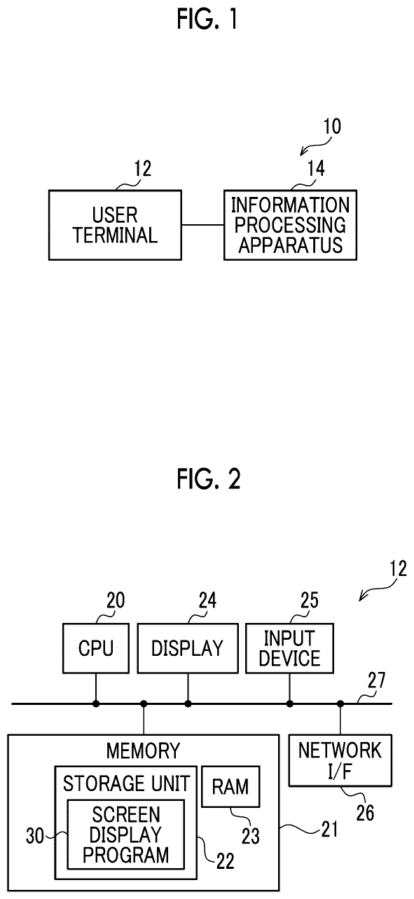

First, a configuration of an information processing system 10 according to the present embodiment will be described with reference to FIG. 1. As illustrated in FIG. 1, the information processing system 10 includes a user terminal 12 used by a user such as a doctor and an information processing apparatus 14. Examples of the user terminal 12 include a tablet computer and a personal computer. Examples of the information processing apparatus 14 include a server computer and a cloud server. The user terminal 12 and the information processing apparatus 14 are examples of a document creation support apparatus according to the disclosed technology.

Each of the user terminal 12 and the information processing apparatus 14 is connected to a network and can transmit and receive data with each other.

Next, a hardware configuration of the user terminal 12 according to the present embodiment will be described with reference to FIG. 2. As illustrated in FIG. 2, the user terminal 12 includes a central processing unit (CPU) 20, a memory 21, a display 24, an input device 25, and a network interface (I/F) 26.

The CPU 20 implements various functions by executing a program stored in a storage unit 22, described later. The CPU 20 is an example of a processor according to the disclosed technology.

The memory 21 includes the storage unit 22 and a random access memory (RAM) 23.

The RAM 23 is a memory for primary storage and is, for example, a RAM such as a static random access memory (SRAM) or a dynamic random access memory (DRAM).

The storage unit 22 is a non-volatile memory and is implemented by, for example, at least one of a hard disk drive (HDD), a solid state drive (SSD), or a flash memory. A screen display program 30 is stored in the storage unit 22 as a storage medium. The CPU 20 reads out the screen display program 30 from the storage unit 22, loads the screen display program 30 into the RAM 23, and executes the loaded screen display program 30.

The screen display program 30 is a program for displaying various screens on the display 24 based on data received from the information processing apparatus 14 through the network I/F 26. The screen display program 30 includes, for example, a web browser program.

The display 24 is a device that displays various screens and is, for example, a liquid crystal display or an electro luminescence (EL) display. The input device 25 is a device for a user to provide input and is, for example, at least any of a keyboard, a mouse, a microphone for audio input, a touchpad for proximity input including a contact, or a camera for gesture input. The network I/F 26 is an interface for connecting to a network. A bus 27 connects the CPU 20, the memory 21, the display 24, the input device 25, and the network I/F 26 to each other.

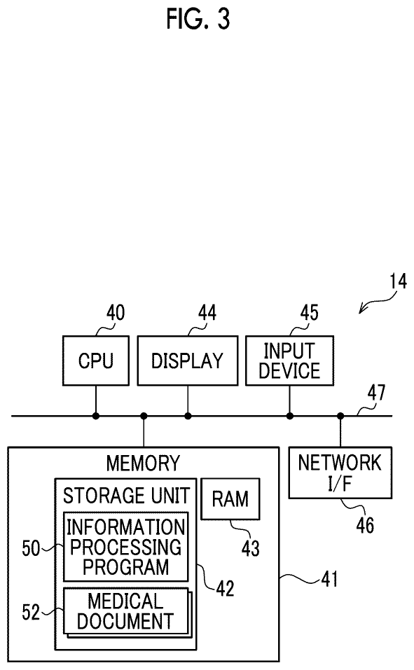

Next, a hardware configuration of the information processing apparatus 14 according to the present embodiment will be described with reference to FIG. 3. As illustrated in FIG. 3, the information processing apparatus 14 includes a CPU 40, a memory 41, a display 44, an input device 45, and a network I/F 46.

The CPU 40 implements various functions by executing a program stored in a storage unit 42, described later. The CPU 40 is an example of a processor according to the disclosed technology.

The memory 41 includes the storage unit 42 and a RAM 43. The RAM 43 is a memory for primary storage and is, for example, a RAM such as an SRAM or a DRAM.

The storage unit 42 is a non-volatile memory and is implemented by, for example, at least one of an HDD, an SDD, or a flash memory. An information processing program 50 is stored in the storage unit 42 as a storage medium. The CPU 40 reads out the information processing program 50 from the storage unit 42, loads the information processing program 50 into the RAM 43, and executes the loaded information processing program 50.

In addition, a plurality of medical documents 52 created by the user are stored in the storage unit 42. The medical document 52 is an example of a reference document to be used for creating a subject document. In the present embodiment, the subject document may be a document such as a new document or a document being edited. The subject document may be any document as long as being editable by adding characters. In the present embodiment, an example in which a summary document such as a hospital discharge summary is applied as the subject document will be described. Examples of the medical document 52 include an electronic medical record. The medical document 52 is created for each patient, and identification information such as a patient ID of the patient is attached to the medical document 52 as attribute information. Accordingly, which patient is related can be identified. Which patient is related may also be identifiable by inputting the patient ID in a specific input field in the document or storing the medical document 52 in a folder for each patient. Similarly, a creation date of the medical document 52 can also be identified. The medical document 52 is not limited to the electronic medical record and may be a radiology report, a medical fee invoice called a receipt, or the like. In addition, the reference document is not limited to the medical document 52 and may be a document, other than the medical document 52, to be used for creating the summary document. In addition, the subject document is not limited to the summary document and may be the medical document 52 that is newly created using the plurality of created medical documents 52. In addition, the subject document may be a document such as a research diary created using the plurality of created medical documents 52. In addition, while a case where the medical document 52 is stored in the storage unit 42 of the information processing apparatus 14 has been illustratively described in the present embodiment, the disclosed technology is not limited to this aspect. For example, the medical document 52 may be stored in a separate data storage server and be acquired through a network.

The display 44 is a device that displays various screens and is, for example, a liquid crystal display or an EL display. The input device 45 is a device for the user to provide input and is, for example, at least any of a keyboard, a mouse, a microphone for audio input, a touchpad for proximity input including a contact, or a camera for gesture input. The network I/F 46 is an interface for connecting to a network. A bus 47 connects the CPU 40, the memory 41, the display 44, the input device 45, and the network I/F 46 to each other.

The user such as a doctor creates the medical document 52 such as the electronic medical record daily with respect to a hospitalized patient and creates a subject document called the hospital discharge summary at the specific time such as the time of hospital discharge by summarizing the plurality of medical documents 52 created during a hospitalization period.

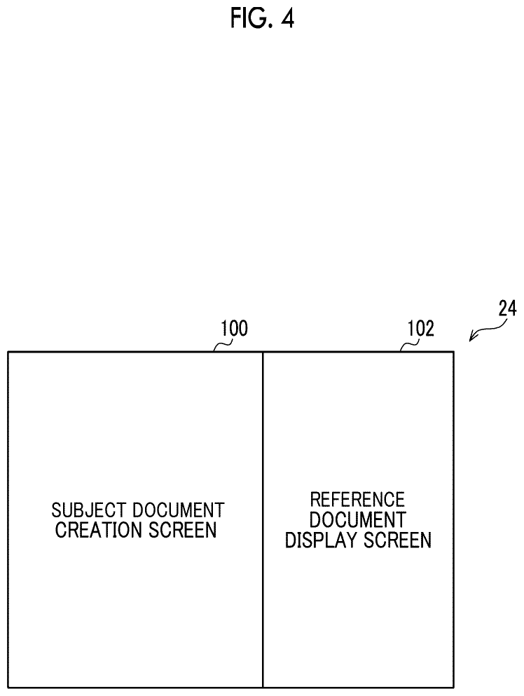

As illustrated in FIG. 4, the information processing system 10 according to the present embodiment displays a subject document creation screen 100 for creating the subject document and a reference document display screen 102 for displaying the medical document 52 as an example of a reference document to be used for creating the subject document, on the display 24 of the user terminal 12. Accordingly, the information processing system 10 supports the user in creating the subject document.

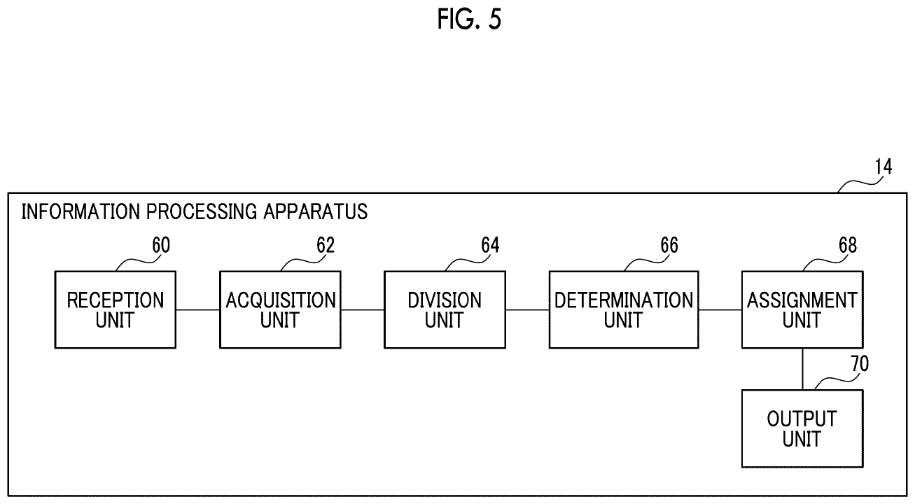

Next, a functional configuration of the information processing apparatus 14 will be described with reference to FIG. 5. As illustrated in FIG. 5, the information processing apparatus 14 includes a reception unit 60, an acquisition unit 62, a division unit 64, a determination unit 66, an assignment unit 68, and an output unit 70. The CPU 40 functions as the reception unit 60, the acquisition unit 62, the division unit 64, the determination unit 66, the assignment unit 68, and the output unit 70 by executing the information processing program 50.

The reception unit 60 receives identification information of a patient (hereinafter, referred to as a “target patient”) as a target for creating the subject document and a period (hereinafter, referred to as a “target period”) as a target for creating the subject document. Examples of the target period include a hospitalization period in which the patient is hospitalized once. For example, the identification information of the target patient and the target period are input into the user terminal 12 by the user and are transmitted to the information processing apparatus 14 from the user terminal 12. Target periods may be stored in association with the identification information of the patients, and the target period corresponding to the patient may be derived in accordance with the input of the identification information of the patient provided by the user.

The acquisition unit 62 acquires the plurality of medical documents 52 created for the target patient in the target period from the storage unit 42 using the identification information of the target patient and the target period received by the reception unit 60.

The division unit 64 divides each of the plurality of medical documents 52 acquired by the acquisition unit 62 into a plurality of partial documents. In the present embodiment, the division unit 64 divides the medical document 52 into a plurality of partial documents in units of one sentence. A division unit of the division unit 64 is not limited to the unit of one sentence and may be a unit of a plurality of sentences or a unit of a paragraph.

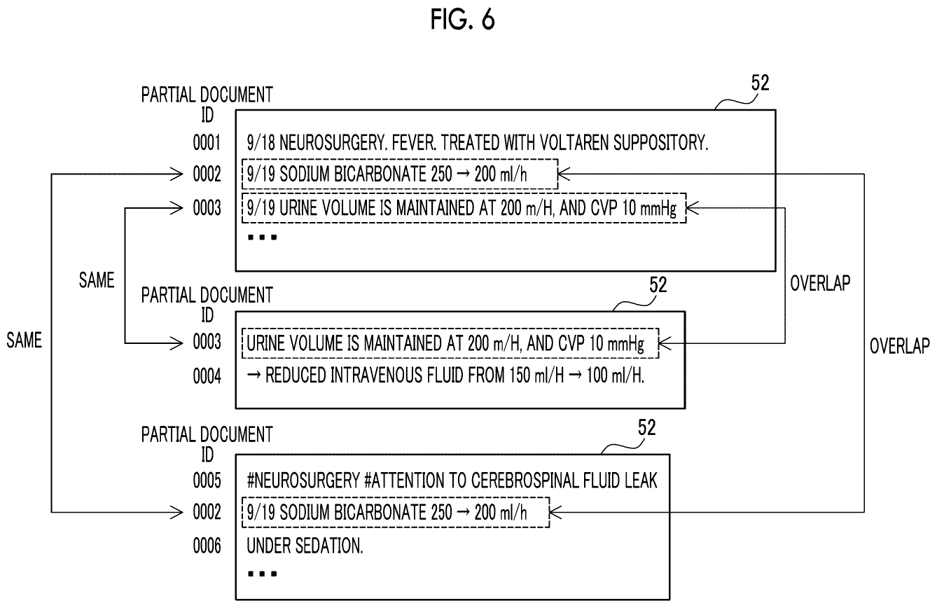

As illustrated in FIG. 6, the determination unit 66 determines whether or not each partial document divided by the division unit 64 overlaps with other partial documents. Specifically, the determination unit 66 selects each partial document in order as a partial document of an overlap determination target and determines whether or not the selected partial document of the overlap determination target overlaps with a partial document other than the overlap determination target. A combination of the partial document of the overlap determination target and the partial document other than the overlap determination target includes a combination included in different medical documents 52 and a combination included in the same medical document 52.

In the present embodiment, the determination unit 66 derives a ratio of match between two partial documents and, in a case where the derived ratio of match is greater than or equal to a threshold value, determines that the two partial documents used for deriving the ratio of match include an overlapping part. An evaluation indicator such as a recall-oriented understudy for gisting evaluation (ROUGE) score for evaluating the ratio of match between two texts can be applied as the ratio of match between the two partial documents. That is, even in a case where the two partial documents do not completely match as in “9/19 urine volume maintained at 200 m/H, CVP 10 mmHg” and “urine volume maintained at 200 m/H, CVP 10 mmHg” in the example illustrated in FIG. 6, it may be determined that the two partial documents overlap with each other. The threshold value used for the overlap determination may be adjustable by the user.

As illustrated in FIG. 6, the determination unit 66 assigns a partial document ID as an example of identification information for identifying the partial document to each partial document. At this point, the determination unit 66 assigns the same partial document ID to a plurality of partial documents determined as overlapping with each other.

For example, the same texts having different meanings may be used for a relatively short text such as “refer to diagram below”. Therefore, the determination unit 66 may exclude a partial document having a certain number of characters or less from the overlap determination target.

In addition, for example, in a case where the two partial documents indicate an examination item and a numerical value of an examination result of the examination item, and one partial document is “value A is 0.2” and the other partial document is “value A is 0.8”, the ratio of match between the two partial documents is a relatively high value. However, even in a case where parts indicating the examination item match, the two partial documents are considered to be different from each other in a case where numerical value information indicating the examination result is different from each other. Therefore, even in a case where the ratio of match between the two partial documents is greater than or equal to the threshold value, the determination unit 66 may determine that the two partial documents used for deriving the ratio of match do not include an overlapping part, that is, do not overlap with each other, in a case where the numerical value information included in each of the two partial documents is different from each other.

Similarly, for example, in a case where the two partial documents indicate the examination item and evaluation of the examination result of the examination item, and one partial document is “value A is high” and the other partial document is “value A is low”, the ratio of match between the two partial documents is a relatively high value. However, even in a case where the parts indicating the examination item match, the two partial documents are considered to be different from each other in a case where keywords indicating the evaluation of the examination result are different from each other. Therefore, even in a case where the ratio of match between the two partial documents is greater than or equal to the threshold value, the determination unit 66 may determine that the two partial documents used for deriving the ratio of match do not include an overlapping part, that is, do not overlap with each other, in a case where specific keywords included in each of the two partial documents are different from each other. Examples of the specific keyword in this case include a keyword indicating a change in a state or a change in a numerical value or a keyword such as “high” and “low”, “rise” and “fall”, and “positive” and “negative” indicating a determination result with respect to a reference value.

In addition, the determination unit 66 may assign a weight to a specific keyword in deriving the ratio of match between the two partial documents. For example, in a case where the number of matching words is used as the ratio of match, the determination unit 66 assigns the weight to a word corresponding to the specific keyword such that the ratio of match is increased, by counting 1.5 instead of 1. Examples of the specific keyword in this case include a disease name included in a medical dictionary and a name of treatment such as surgery.

The assignment unit 68 assigns a degree of importance (hereinafter, referred to as a “first degree of importance”) to each of the plurality of medical documents 52 acquired by the acquisition unit 62. That is, each of the plurality of medical documents 52 acquired by the acquisition unit 62 is associated with the first degree of importance. As the first degree of importance, the assignment unit 68 may assign a degree of importance designated by the user or assign a degree of importance in accordance with a keyword or the like included in the medical document 52.

In addition, the assignment unit 68 assigns a degree of importance (hereinafter, referred to as a “second degree of importance”) to each of the plurality of partial documents obtained through division processing performed by the division unit 64. That is, each of the plurality of partial documents obtained through the division processing performed by the division unit 64 is associated with the second degree of importance. As the second degree of importance, the assignment unit 68 may assign a degree of importance designated by the user or assign a degree of importance in accordance with a keyword or the like included in the partial document.

The output unit 70 outputs the plurality of medical documents 52 acquired by the acquisition unit 62 to the user terminal 12 through the network I/F 46. At this point, the output unit 70 also outputs assignment information of the medical document 52 to the user terminal 12 through the network I/F 46. Examples of the assignment information include the partial document ID, the first degree of importance, and the second degree of importance described above.

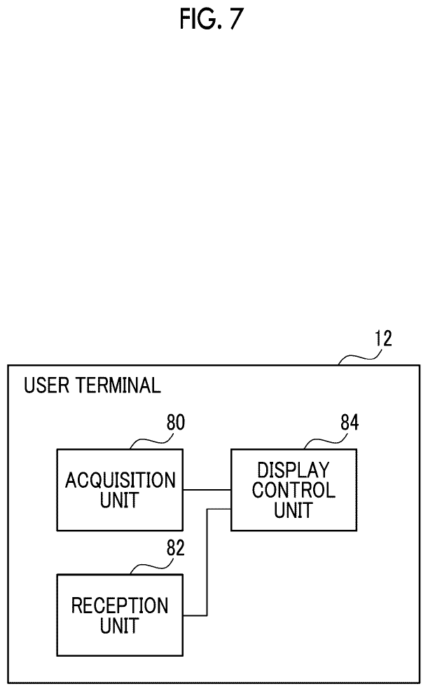

Next, a functional configuration of the user terminal 12 will be described with reference to FIG. 7. As illustrated in FIG. 7, the user terminal 12 includes an acquisition unit 80, a reception unit 82, and a display control unit 84. The CPU 20 functions as the acquisition unit 80, the reception unit 82, and the display control unit 84 by executing the screen display program 30.

The acquisition unit 80 acquires the plurality of medical documents 52 transmitted from the information processing apparatus 14 through the network I/F 26.

The reception unit 82 receives various operations of the user through the input device 25. For example, the reception unit 82 receives selection of the medical document 52 by the user on the reference document display screen 102.

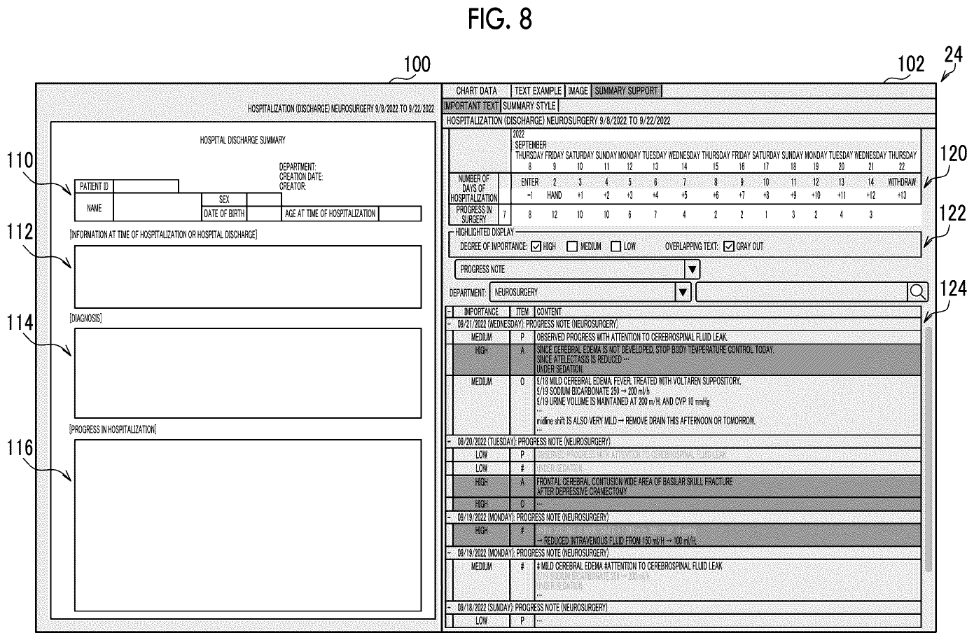

As illustrated in FIG. 8, the display control unit 84 displays the subject document creation screen 100 for creating the subject document and the reference document display screen 102 for displaying the medical document 52 to be used for creating the subject document on the display 24. The subject document creation screen 100 includes an input field 110 of information about the target patient, an input field 112 of information at a time of hospitalization and a time of hospital discharge of the target patient, an input field 114 of a diagnosis result of the target patient such as a disease name, and an input field 116 of a progress in hospitalization of the target patient.

The reference document display screen 102 includes a display region 120 in which information related to the target period is displayed, a display region 122 in which information for the user to set highlighted display, and a display region 124 in which the plurality of medical documents 52 are displayed.

For example, in the display region 120, dates in the target period, the number of days of hospitalization, the number of elapsed days counted from a surgery day as a starting day, and the number of medical documents 52 created on each day in the target period are displayed.

For example, in the display region 122, a checkbox for designating the first degree of importance of the medical document 52 displayed in a highlighted manner and a checkbox for designating whether or not to display an overlapping text in a non-highlighted manner are displayed. As illustrated in the example in FIG. 8, the user designates the overlapping text as being displayed in a non-highlighted manner by placing a check in the checkbox for the overlapping text.

In a case where the plurality of medical documents 52 are displayed in the display region 124 of the reference document display screen 102, the display control unit 84 displays the overlapping part included in the medical documents 52 in a non-highlighted manner with respect to a non-overlapping part. The overlapping part according to the present embodiment means a partial document determined as overlapping, and the non-overlapping part means a partial document determined as not overlapping.

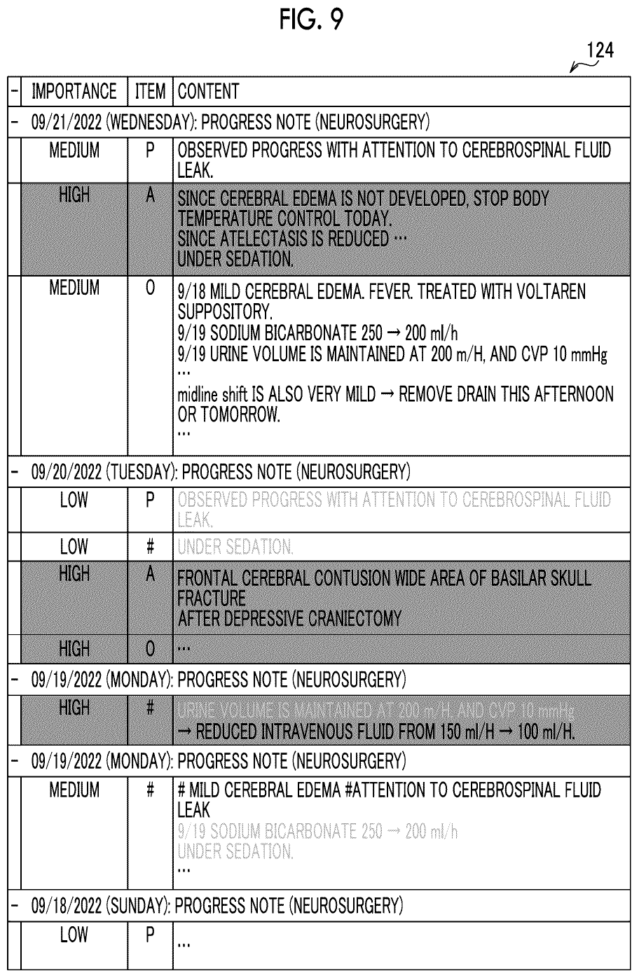

In this case, as illustrated in FIG. 9, the display control unit 84, for the partial document determined as overlapping, displays the partial document displayed later in a non-highlighted manner with respect to the partial document displayed earlier in accordance with a display order of the plurality of medical documents 52 in the display region 124. In the example in FIG. 9, a display order such that a display position of the medical document 52 is higher as the creation date of the medical document 52 is newer is applied as the display order of the plurality of medical documents 52. That is, this display order means an order of display positions. The display order is not limited to the order of the newest date and may be, for example, other orders such as an order of the oldest date or an order of the highest first degree of importance. Accordingly, one partial document in a combination of partial documents determined as overlapping is not displayed in a non-highlighted manner with respect to the partial document determined as not overlapping, and the partial documents other than the one partial document are displayed in a non-highlighted manner with respect to the partial document determined as not overlapping. In the present embodiment, the first partial document in the display order is not displayed in a non-highlighted manner and has the same display aspect as the non-overlapping part, and the second partial document or later in the display order is displayed in a non-highlighted manner.

In addition, in the example in FIG. 9, the plurality of medical documents 52 are displayed in a table form. One row of the table corresponds to one medical document 52. An importance column indicates the first degree of importance assigned to the medical document 52, and a content column indicates content of the medical document 52. In addition, in the example in FIG. 9, an item column indicates a category of the medical document 52. Examples of the category include “Subject (subject information)”, “Object (object information)”, “Assessment (evaluation)”, and “Plan (schedule)”. In the example in FIG. 9, the item column describes an initial of a word indicating a corresponding category. In addition, the item column also describes “#” corresponding to a problem list. Here, a problem includes at least any of a medical abnormal event (illness) recognized in the patient by the doctor, a disease, a simple opinion, a past medical history, a social event, or the like.

As illustrated in FIG. 9, “under sedation.” that is one of the partial documents included in the medical document 52 which is later in the display order and which is created on “2022 Sep. 20” is displayed in a non-highlighted manner with respect to “under sedation.” that is one of the partial documents included in the medical document 52 which is earlier in the display order and which is created on “2022 Sep. 21”.

Here, displaying in a non-highlighted manner is displaying in an unnoticeable manner and, for example, means displaying the text in a display color (for example, gray) set as an unnoticeable color with respect to a display color (for example, black) as a reference. In the example in FIG. 9, “under sedation.” included in the medical document 52 created on “2022 Sep. 21” is displayed in black, and “under sedation.” included in the medical document 52 created on “2022 Sep. 20” is displayed in gray.

The display control unit 84 can determine whether or not each partial document included in the plurality of medical documents 52 is the overlapping partial document by determining whether or not the partial document ID assigned to each partial document is the same.

For example, a new medical document 52 may be created by adding new content after copying the content of the immediately previously created medical document 52. In this case, the medical document 52 includes a relatively large amount of the overlapping part. At this point, for example, in a case where only one partial document among 20 partial documents included in the medical document 52 is determined as overlapping, it is relatively highly likely that the one partial document coincidentally matches. Therefore, in a case where a ratio of an amount of the partial document determined as overlapping to an amount of the medical document 52 is less than or equal to a threshold value, the display control unit 84 may not display the partial document that is included in the medical document 52 and that is determined as overlapping in a non-highlighted manner with respect to the partial document determined as not overlapping. Here, not displaying in a non-highlighted manner means, for example, displaying the partial document determined as overlapping in the same display aspect as the partial document determined as not overlapping.

In addition, in the medical document 52 including a relatively large amount of the overlapping part, display of the overlapping part may distract the user. Therefore, in a case where the ratio of the amount of the partial document determined as overlapping to the amount of the medical document 52 is greater than or equal to the threshold value, the display control unit 84 may not display at least a part of the partial document that is included in the medical document 52 and that is determined as overlapping. In addition, for example, in a case where there are a certain number or more of consecutive partial documents determined as overlapping in the medical document 52, the display control unit 84 may not display at least a part of the consecutive partial documents. In addition, for example, in a case where the number of characters of the partial document determined as overlapping in the medical document 52 is greater than or equal to a threshold value, the display control unit 84 may not display at least a part of the partial document, for example, characters corresponding to the threshold value or more.

In addition, in a case where the plurality of medical documents 52 are displayed in the display region 124, the display control unit 84 may not display at least a part of the medical document 52 for which all partial documents included in the medical document 52 are determined as overlapping. In this case, the display control unit 84 may not display the entire medical document 52 of which all partial documents are determined as overlapping, or may display the first partial document or the first word of the medical document 52 and not display a part other than the first partial document or the first word.

In addition, as illustrated in FIG. 9, in a case where the plurality of medical documents 52 are displayed in the display region 124, the display control unit 84 may display the medical document 52 of which the first degree of importance is relatively high in a highlighted manner with respect to the medical document 52 of which the first degree of importance is relatively low. For example, the display control unit 84 may display the medical document 52 of which the first degree of importance is greater than or equal to a threshold value in a highlighted manner with respect to the medical document 52 of which the first degree of importance is less than the threshold value. In addition, for example, the display control unit 84 may display a predetermined number of medical documents 52 in an order from the highest first degree of importance in a highlighted manner with respect to the medical document 52 of which the first degree of importance is lower than those of the predetermined number of medical documents 52. Here, displaying in a highlighted manner is displaying in a noticeable manner and, for example, means displaying in a color (for example, light green) set as a noticeable color with respect to a color (for example, white) based on a background color. In the example in FIG. 9, the first degree of importance is represented in three levels of “high”, “medium”, and “low”, and the medical document 52 of which the first degree of importance is “high” is displayed in a highlighted manner with respect to the medical documents 52 of which the first degrees of importance are “medium” and “low”.

In addition, as illustrated in FIG. 9, the display control unit 84 may display the overlapping part included in the medical document 52 of which the first degree of importance is relatively high in a non-highlighted manner with respect to the non-overlapping part. In the example in FIG. 9, for the medical document 52 of which the first degree of importance is “high” on “2022 Sep. 19”, the medical document 52 is displayed in a highlighted manner, and the partial document of the first row determined as overlapping is displayed in a non-highlighted manner.

At this point, the display control unit 84 may prioritize the highlighted display based on the first degree of importance over the non-highlighted display of the overlapping part. Specifically, the display control unit 84 may not display the overlapping part included in the medical document 52 of which the first degree of importance is relatively high in a non-highlighted manner with respect to the non-overlapping part.

In addition, as illustrated in FIG. 9, in a case where the plurality of medical documents 52 are displayed in the display region 124, the display control unit 84 may display the partial document of which the second degree of importance is relatively high in a highlighted manner with respect to the partial document of which the second degree of importance is relatively low. For example, the display control unit 84 may display the partial document of which the second degree of importance is greater than or equal to a threshold value in a highlighted manner with respect to the partial document of which the second degree of importance is less than the threshold value. In addition, for example, the display control unit 84 may display a predetermined number of partial documents in an order from the highest second degree of importance in a highlighted manner with respect to the partial document of which the second degree of importance is lower than those of the predetermined number of partial documents. Here, displaying in a highlighted manner is displaying in a noticeable manner and, for example, means displaying the text in a display color (for example, red) set as a noticeable color with respect to a display color (for example, black) as a reference. In the example in FIG. 9, the second degree of importance is represented in two levels of “high” and “low”. A row described with a bold font is the partial document of which the second degree of importance is “high”, and a row described with a thin font is the partial document of which the second degree of importance is “low”. In addition, in FIG. 9, a text described with the bold font mean a text in red.

In addition, for the partial document of which the second degree of importance is relatively high, the display control unit 84 may display the partial document determined as overlapping in a non-highlighted manner with respect to the partial document determined as not overlapping. In this case, the non-highlighted display based on the overlap determination is prioritized over the highlighted display based on the second degree of importance.

In addition, the display control unit 84 may not display the partial document of which the second degree of importance is relatively high and that is determined as overlapping in a non-highlighted manner with respect to the partial document determined as not overlapping. In this case, the highlighted display based on the second degree of importance is prioritized over the non-highlighted display based on the overlap determination.

In addition, the display control unit 84 may implement the highlighted display and the non-highlighted display by varying a size of the text, a thickness of a line of the text, and the like, in addition to implementing the highlighted display and the non-highlighted display by varying the color of the text and the background color.

In addition, in order to identify a combination of the second degree of importance and whether or not overlapping occurs in each partial document, the display control unit 84 may vary the display aspect in accordance with the combination of the second degree of importance and whether or not overlapping occurs. In this case, the display control unit 84 may display the partial document of which the second degree of importance is “high” and that is determined as not overlapping in a highlighted manner with a red font and display the partial document with the bold font. In addition, in this case, the display control unit 84 may display the partial document of which the second degree of importance is “high” and that is determined as overlapping in a highlighted manner with the red font and display the partial document in a non-highlighted manner with the thin font. In addition, in this case, the display control unit 84 may display the partial document of which the second degree of importance is “low” and that is determined as not overlapping with a black font and display the partial document with the thin font. In addition, in this case, the display control unit 84 may display the partial document of which the second degree of importance is “low” and that is determined as overlapping in a non-highlighted manner in gray and display the partial document with the thin font.

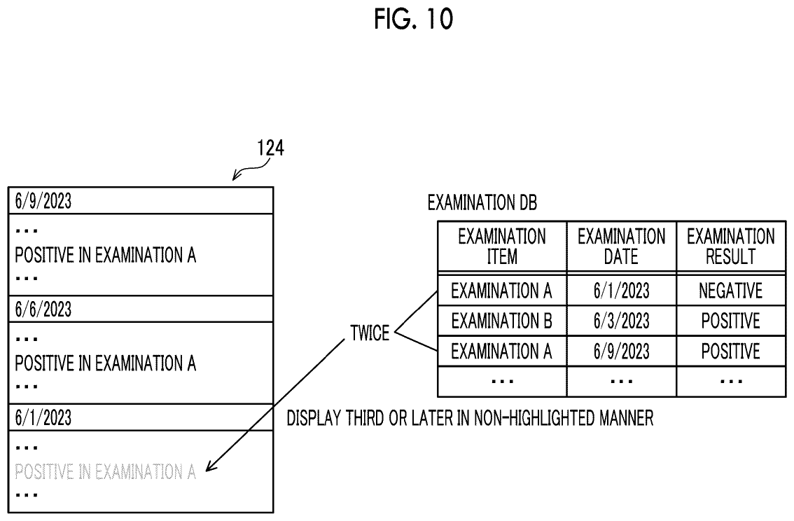

In addition, as illustrated in FIG. 10, in a case where the partial document determined as overlapping includes the examination item or a medical practice name (for example, the name of treatment such as surgery), the display control unit 84 may display, for the partial document, the partial document displayed later in order than a point in order corresponding to the number of executions of an examination indicated by the examination item or a medical practice indicated by the medical practice name in a non-highlighted manner with respect to the partial document displayed earlier in order in accordance with the display order of the plurality of medical documents 52. In this case, for example, the display control unit 84 may refer to data in which the examination item is associated with an execution date of the examination for each patient and derive the number of executions of the examination executed on the target patient in the target period. FIG. 10 illustrates an example in which “examination A” is performed twice in the target period, there are three partial documents that are determined as overlapping and that include “examination A”, the first and second partial documents in the display order are not displayed in a non-highlighted manner, and the third partial document or later in the display order is displayed in a non-highlighted manner.

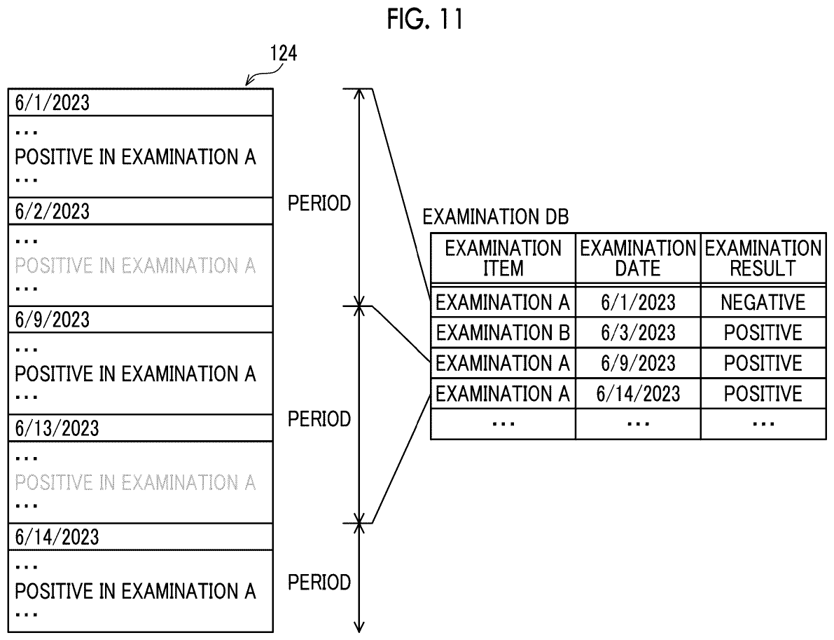

In addition, as illustrated in FIG. 11, in a case where the partial document determined as overlapping includes the examination item or the medical practice name, the display control unit 84 may divide the target period into a plurality of periods by separating the target period on an execution date of the examination indicated by the examination item or the medical practice indicated by the medical practice name. In this case, for the partial document that is determined as overlapping and that includes the examination item or the medical practice name in each period, the display control unit 84 may display the partial document displayed later in a non-highlighted manner with respect to the partial document displayed earlier in accordance with the display order of the plurality of medical documents 52. FIG. 11 illustrates an example in which, in each period, the first partial document in the display order is not displayed in a non-highlighted manner and has the same display aspect as the non-overlapping part, and the second partial document or later in the display order is displayed in a non-highlighted manner.

In addition, for the partial document determined as overlapping, the display control unit 84 may vary the display aspect of the partial document in accordance with the number of overlapping partial documents. For example, for the partial document for which the number of overlapping partial documents is less than a threshold value, the display control unit 84 may set the first partial document in the display order to have the same display aspect as the non-overlapping part and display the second partial document or later in the display order in a non-highlighted manner with respect to the non-overlapping part. In this case, for the partial document for which the number of overlapping partial documents is greater than or equal to the threshold value, the display control unit 84 may display the first partial document in the display order in a highlighted manner with respect to the non-overlapping part and display the second partial document or later in the display order in a non-highlighted manner with respect to the non-overlapping part.

In addition, for example, for the partial document for which the number of overlapping partial documents is less than the threshold value, the display control unit 84 may display the first partial document in the display order in a highlighted manner with respect to the non-overlapping part and display the second partial document or later in the display order in a non-highlighted manner (for example, display in a gray text color) with respect to the non-overlapping part. In this case, for the partial document for which the number of overlapping partial documents is greater than or equal to the threshold value, the display control unit 84 may display the second partial document or later in the display order in a non-highlighted manner with respect to the non-overlapping part and set a degree of the non-highlighted display to be different from that in a case where the number of overlapping partial documents is less than the threshold value. In the second partial document or later in this case, the text is displayed with the bold font in gray.

The display control unit 84 copies at least a part of the medical document 52 selected on the reference document display screen 102 to the subject document creation screen 100. For example, the display control unit 84 may copy the entire medical document 52 selected on the reference document display screen 102 to the input field 116 of the subject document creation screen 100.

In addition, in a case where the medical document 52 selected on the reference document display screen 102 includes the overlapping part, the display control unit 84 may copy a part of the selected medical document 52 excluding the overlapping part to the subject document creation screen.

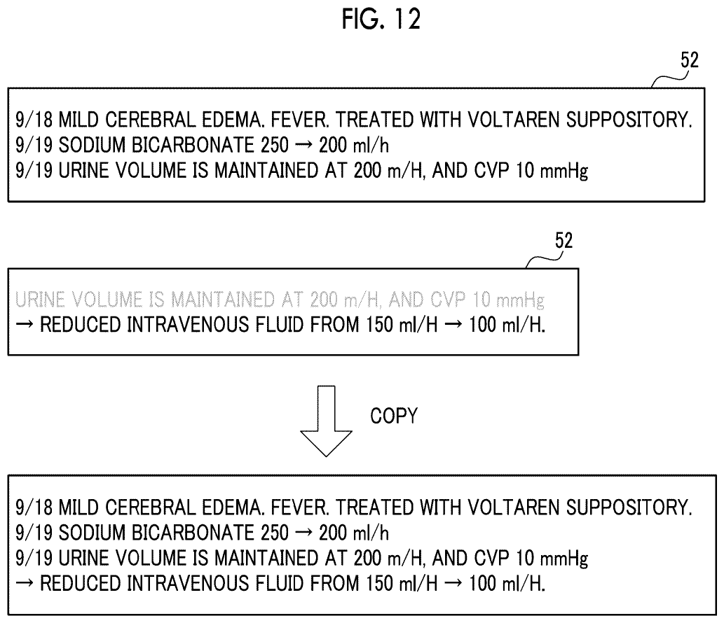

As illustrated in FIG. 12, in a case where a plurality of medical documents 52 on the reference document display screen 102 are selected, and each of the reference documents of the plurality of selected medical documents 52 includes the same overlapping part, the display control unit 84 may perform copying as follows. That is, in this case, the display control unit 84 may copy a part of one of the plurality of selected medical documents 52 other than the same overlapping part excluding the same overlapping part to the subject document creation screen 100. FIG. 12 illustrates an example in which among the partial documents determined as overlapping, the partial document earlier in the display order is copied, and the partial document later in the display order, that is, the partial document displayed in a non-highlighted manner, is not copied. The plurality of selected medical documents 52 in this case include the plurality of medical documents 52 selected at the same time and the plurality of medical documents 52 selected in order. In a case where the plurality of medical documents 52 are selected in order, a part other than the overlapping part of the medical document 52 selected later is copied in a case where the medical document 52 selected later includes the overlapping part with the already copied medical document 52.

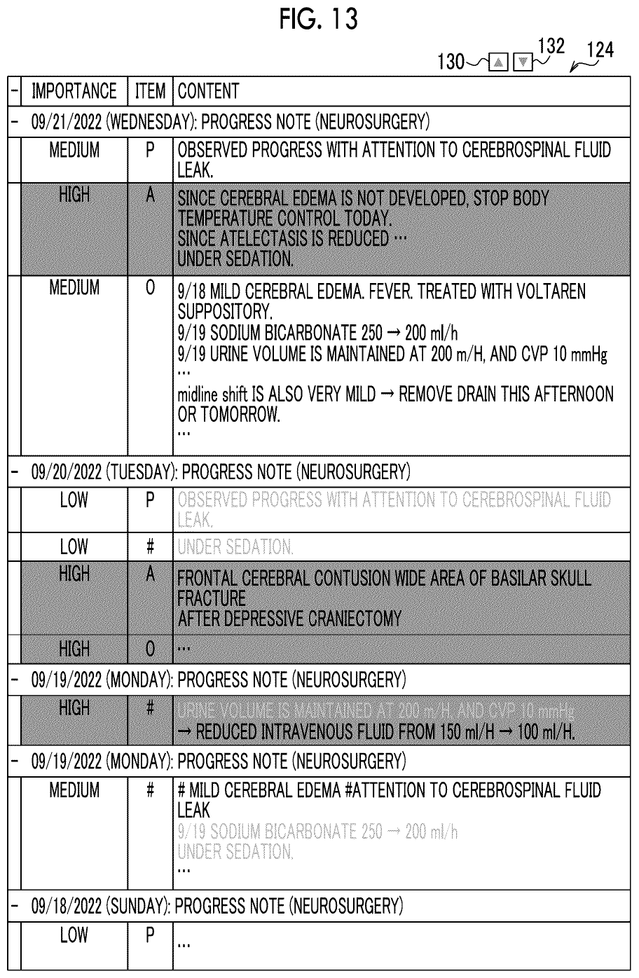

In addition, as illustrated in FIG. 13, the display control unit 84 may display the medical documents 52 including the same overlapping part in a state where the user can perform an operation of tracking the same overlapping part, by displaying a return button 130 and an advance button 132 in the display region 124. For example, the user selects one of the partial documents determined as overlapping by clicking or dragging the partial document. In addition, for example, the user selects one of the partial documents determined as overlapping including a keyword by searching with the keyword. Then, in a case where the user designates the advance button 132, the display control unit 84 updates display to a state where the partial document that is determined as overlapping with the selected partial document and that is later in the display order is selected. Meanwhile, in a case where the user designates the return button 130, the display control unit 84 updates display to a state where the partial document that is determined as overlapping with the selected partial document and that is earlier in the display order is selected. Accordingly, the user can track the selected overlapping part.

In addition, the display control unit 84 may display a list of the medical documents 52 including the same overlapping part on the reference document display screen 102. For example, the display control unit 84 may display a list of the content of the medical documents 52 including the same overlapping part or display a list of the attribute information, such as a title or the creation date, of the medical documents 52. In addition, the list in this case may be displayed as a pop-up.

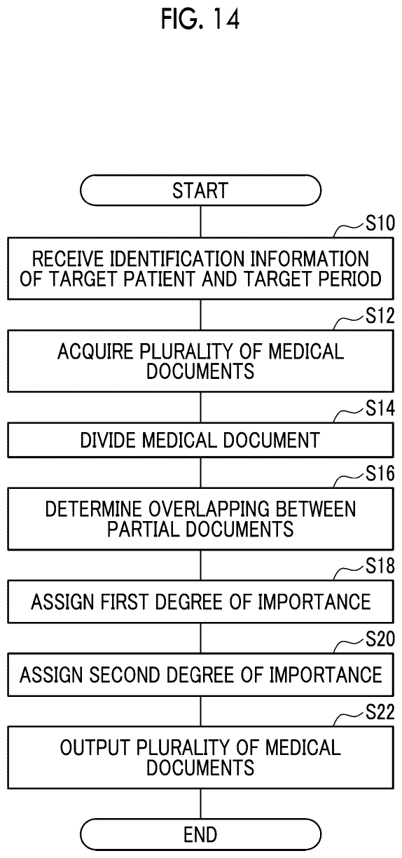

Next, an action of the information processing apparatus 14 will be described with reference to FIG. 14. Overlap determination processing illustrated in FIG. 14 is executed by executing the information processing program 50 via the CPU 40. For example, the overlap determination processing illustrated in FIG. 14 is executed in a case where an execution start instruction is input by the user.

In step S10 in FIG. 14, the reception unit 60 receives the identification information of the target patient and the target period. In step S12, the acquisition unit 62 acquires the plurality of medical documents 52 created for the target patient in the target period from the storage unit 42 using the identification information of the target patient and the target period received in step S10. In step S14, the division unit 64, as described above, divides each of the plurality of medical documents 52 acquired in step S12 into a plurality of partial documents.

In step S16, the determination unit 66, as described above, determines whether or not each partial document divided in step S14 overlaps with other partial documents. In step S18, the assignment unit 68 assigns the first degree of importance to each of the plurality of medical documents 52 acquired in step S10. In step S20, the assignment unit 68 assigns the second degree of importance to each of the plurality of partial documents obtained through the division processing in step S14.

In step S22, the output unit 70 outputs the plurality of medical documents 52 acquired in step S10 to the user terminal 12 through the network I/F 46. At this point, the output unit 70 also outputs the assignment information of the medical document 52 to the user terminal 12 through the network I/F 46. In a case where the processing in step S22 is finished, the overlap determination processing is finished. In the present overlap determination processing, at least one of step S18 or step S20 may not be executed.

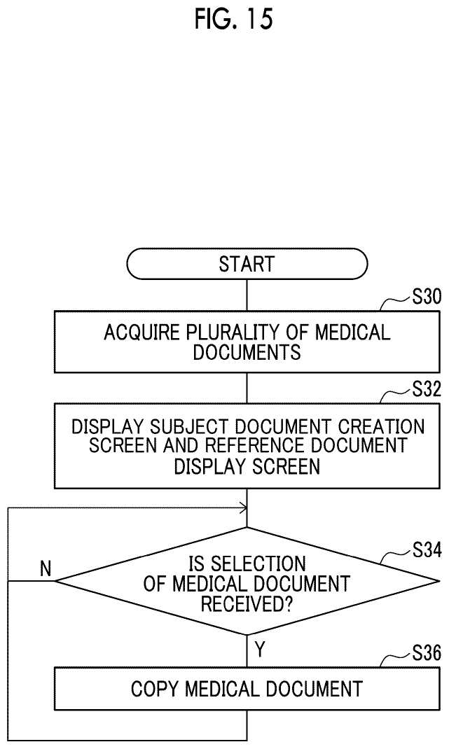

Next, an action of the user terminal 12 will be described with reference to FIG. 15. Display control processing illustrated in FIG. 15 is executed by executing the screen display program 30 via the CPU 20. For example, the display control processing illustrated in FIG. 15 is executed in a case where the user terminal 12 receives the plurality of medical documents 52 transmitted from the information processing apparatus 14 through the processing in step S22.

In step S30 in FIG. 15, the acquisition unit 80 acquires the plurality of medical documents 52 transmitted from the information processing apparatus 14 through the network I/F 26. In step S32, the display control unit 84 displays the subject document creation screen 100 for creating the subject document and the reference document display screen 102 for displaying the medical document 52 to be used for creating the subject document on the display 24. At this point, the display control unit 84, as described above, displays the overlapping part included in the medical document 52 in a non-highlighted manner with respect to the non-overlapping part.

In step S34, the reception unit 82 waits until receiving selection of the medical document 52 by the user on the reference document display screen 102. In a case where the reception unit 82 receives selection of the medical document 52 by the user on the reference document display screen 102, the processing transitions to step S36. In step S36, the display control unit 84, as described above, copies at least a part of the medical document 52 selected on the reference document display screen 102 to the subject document creation screen 100. In a case where the processing in step S36 is finished, the processing returns to step S34. The display control processing is finished in a case where, for example, the user performs a finish operation through the input device 25.

As described above, according to the present embodiment, creation of a subject document using a plurality of reference documents can be effectively supported.

In the embodiment, at least a part of functional units comprised in the information processing apparatus 14 may be comprised in the user terminal 12, or at least a part of functional units comprised in the user terminal 12 may be comprised in the information processing apparatus 14. For example, the information processing apparatus 14 may generate the subject document creation screen 100 and provide the subject document creation screen 100 to the user terminal 12 in a streaming manner. In addition, each functional unit comprised in the user terminal 12 and in the information processing apparatus 14 may be comprised in one computer.

In addition, while a case where the display control unit 84 displays the subject document creation screen 100 and the reference document display screen 102 when the user creates the subject document has been described in the embodiment, the disclosed technology is not limited to this aspect. For example, the display control unit 84 may display only the reference document display screen 102 when the user refers to the reference documents.

In addition, in the embodiment, for example, the following various processors can be used as a hardware structure of a processing unit that executes various types of processing, such as each functional unit of the user terminal 12 and each functional unit of the information processing apparatus 14. The various processors include, in addition to a CPU that is a general-purpose processor functioning as various processing units by executing software (program) as described above, a programmable logic device (PLD) such as a field programmable gate array (FPGA) that is a processor having a circuit configuration changeable after manufacture, a dedicated electric circuit such as an application specific integrated circuit (ASIC) that is a processor having a circuit configuration dedicatedly designed to execute specific processing, and the like.

One processing unit may be composed of one of the various processors or may be composed of a combination of two or more processors of the same type or different types (for example, a combination of a plurality of FPGAs or a combination of a CPU and an FPGA). In addition, a plurality of processing units may be composed of one processor.

As an example of the plurality of processing units composed of one processor, first, as represented by a computer such as a client and a server, a form of one processor that is composed of a combination of one or more CPUs and software and that functions as the plurality of processing units is possible. Second, as represented by a system on chip (SoC), a form of using a processor that implements functions of the entire system including the plurality of processing units in one integrated circuit (IC) chip is possible. Accordingly, various processing units are configured using one or more of the various processors as the hardware structure.

Furthermore, more specifically, an electric circuit (circuitry) in which circuit elements such as semiconductor elements are combined can be used as the hardware structure of the various processors.

In addition, while an aspect in which the screen display program 30 is stored (installed) in advance in the storage unit 22 has been described in each embodiment, the disclosed technology is not limited to this aspect. The screen display program 30 may be provided in a form of a recording on a recording medium such as a compact disc read only memory (CD-ROM), a digital versatile disc read only memory (DVD-ROM), and a universal serial bus (USB) memory. In addition, the screen display program 30 may be provided in a form of a download from an external apparatus through a network.

In addition, while an aspect in which the information processing program 50 is stored (installed) in advance in the storage unit 42 has been described in the embodiment, the disclosed technology is not limited to this aspect. The information processing program 50 may be provided in a form of a recording on a recording medium such as a CD-ROM, a DVD-ROM, and a USB memory. In addition, the information processing program 50 may be provided in a form of a download from an external apparatus through a network.

The following appendixes are further disclosed with respect to the above embodiment.

### Appendix 1

A document creation support apparatus comprising at least one processor, in which the processor is configured to display a subject document creation screen for creating a subject document and a reference document display screen for displaying a reference document to be used for creating the subject document, and in a case where a plurality of the reference documents are displayed on the reference document display screen, display an overlapping part included in the reference document in a non-highlighted manner with respect to a non-overlapping part.

### Appendix 2

The document creation support apparatus according to Appendix 1, in which the processor is configured to divide each of the plurality of reference documents into a plurality of partial documents, determine whether or not each partial document overlaps with other partial documents, and for a partial document determined as overlapping, display the partial document displayed later in a non-highlighted manner with respect to the partial document displayed earlier in accordance with a display order of the plurality of reference documents.

### Appendix 3

The document creation support apparatus according to Appendix 2, in which the processor is configured to exclude the partial document having a certain number of characters or less from an overlap determination target.

### Appendix 4

The document creation support apparatus according to any one of Appendixes 1 to 3, in which the processor is configured to, in a case where a ratio of an amount of the overlapping part to an amount of the reference document is less than or equal to a threshold value, not display the overlapping part included in the reference document in a non-highlighted manner with respect to the non-overlapping part.

### Appendix 5

The document creation support apparatus according to any one of Appendixes 1 to 3, in which the processor is configured to, in a case where a ratio of an amount of the overlapping part to an amount of the reference document is greater than or equal to a threshold value, not display at least a part of the overlapping part included in the reference document.

### Appendix 6

The document creation support apparatus according to any one of Appendixes 1 to 5, in which each of the plurality of reference documents is associated with a first degree of importance, and the processor is configured to, in a case where the plurality of reference documents are displayed on the reference document display screen, display the reference document of which the first degree of importance is relatively high in a highlighted manner with respect to the reference document of which the first degree of importance is relatively low.

### Appendix 7

The document creation support apparatus according to Appendix 6, in which the processor is configured to display the overlapping part included in the reference document of which the first degree of importance is relatively high in a non-highlighted manner with respect to the non-overlapping part.

### Appendix 8

The document creation support apparatus according to Appendix 6, in which the processor is configured to not display the overlapping part included in the reference document of which the first degree of importance is relatively high in a non-highlighted manner with respect to the non-overlapping part.

### Appendix 9

The document creation support apparatus according to Appendix 2 or 3, in which each of the plurality of partial documents is associated with a second degree of importance, and the processor is configured to, in a case where the plurality of reference documents are displayed on the reference document display screen, display the partial document of which the second degree of importance is relatively high in a highlighted manner with respect to the partial document of which the second degree of importance is relatively low.

### Appendix 10

The document creation support apparatus according to Appendix 9, in which the processor is configured to, for the partial document of which the second degree of importance is relatively high, display the partial document determined as overlapping in a non-highlighted manner with respect to the partial document determined as not overlapping.

### Appendix 11

The document creation support apparatus according to Appendix 9, in which the processor is configured to not display the partial document of which the second degree of importance is relatively high and that is determined as overlapping, in a non-highlighted manner with respect to the partial document determined as not overlapping.

### Appendix 12

The document creation support apparatus according to any one of Appendixes 1 to 11, in which the processor is configured to, in a case where a ratio of match between the reference documents is greater than or equal to a threshold value, determine that the reference document used for deriving the ratio of match includes the overlapping part.

### Appendix 13

The document creation support apparatus according to Appendix 12, in which the processor is configured to, in a case where the ratio of match between the reference documents is greater than or equal to the threshold value and a specific keyword or numerical value information is different between the reference documents, determine that the reference document used for deriving the ratio of match does not include the overlapping part.

### Appendix 14

The document creation support apparatus according to Appendix 12, in which the processor is configured to assign a weight to a specific keyword in deriving a ratio of match between the reference documents.

### Appendix 15

The document creation support apparatus according to any one of Appendixes 1 to 14, in which the processor is configured to, in a case where a plurality of the reference documents are displayed on the reference document display screen, not display at least a part of the reference document for which all partial documents included in the reference document overlap.

### Appendix 16

The document creation support apparatus according to any one of Appendixes 1 to 15, in which the processor is configured to receive selection of the reference document on the reference document display screen, and copy at least a part of the selected reference document to the subject document creation screen.

### Appendix 17

The document creation support apparatus according to Appendix 16, in which the processor is configured to, in a case where the selected reference document includes the overlapping part, copy a part of the selected reference document excluding the overlapping part to the subject document creation screen.

### Appendix 18

The document creation support apparatus according to Appendix 16, in which the processor is configured to receive selection of a plurality of the reference documents on the reference document display screen, and in a case where each of the plurality of selected reference documents includes the same overlapping part, copy a part of one of the selected reference documents other than the same overlapping part excluding the same overlapping part to the subject document creation screen.

### Appendix 19

The document creation support apparatus according to any one of Appendixes 1 to 18, in which the processor is configured to display the reference document including the same overlapping part in a state where a user is capable of performing an operation of tracking the same overlapping part.

### Appendix 20

The document creation support apparatus according to any one of Appendixes 1 to 18, in which the processor is configured to display a list of the reference documents including the same overlapping part.

### Appendix 21

The document creation support apparatus according to Appendix 2 or 3, in which the processor is configured to, in a case where the partial document determined as overlapping includes an examination item or a medical practice name, display, for the partial document, the partial document displayed later in order than a point in order corresponding to the number of executions of an examination indicated by the examination item or a medical practice indicated by the medical practice name in a non-highlighted manner with respect to the partial document displayed earlier in order in accordance with the display order.

### Appendix 22

The document creation support apparatus according to Appendix 2 or 3, in which the processor is configured to, in a case where the partial document determined as overlapping includes an examination item or a medical practice name, divide a period designated as a period as a target for creating the subject document into a plurality of periods by separating the period on an execution date of an examination indicated by the examination item or a medical practice indicated by the medical practice name, and for the partial document that is determined as overlapping and that includes the examination item or the medical practice name in each period, display the partial document displayed later in a non-highlighted manner with respect to the partial document displayed earlier in accordance with the display order of the plurality of reference documents.

### Appendix 23

The document creation support apparatus according to Appendix 2 or 3, in which the processor is configured to, for the partial document determined as overlapping, vary a display aspect of the partial document in accordance with the number of overlapping partial documents.

### Appendix 24

A document creation support method including, via a processor comprised in a document creation support apparatus, displaying a subject document creation screen for creating a subject document and a reference document display screen for displaying a reference document to be used for creating the subject document, and displaying, in a case where a plurality of the reference documents are displayed on the reference document display screen, an overlapping part included in the reference document in a non-highlighted manner with respect to a non-overlapping part.

### Appendix 25

A document creation support program for causing a processor comprised in a document creation support apparatus to execute a process including displaying a subject document creation screen for creating a subject document and a reference document display screen for displaying a reference document to be used for creating the subject document, and displaying, in a case where a plurality of the reference documents are displayed on the reference document display screen, an overlapping part included in the reference document in a non-highlighted manner with respect to a non-overlapping part.

What is claimed is:

1. A document creation support apparatus comprising:
at least one processor,
wherein the processor is configured to:
display a subject document creation screen for creating a subject document and a reference document display screen for displaying a reference document to be used for creating the subject document; and
in a case where a plurality of the reference documents are displayed on the reference document display screen, display an overlapping part included in the reference document in a non-highlighted manner with respect to a non-overlapping part.

2. The document creation support apparatus according to claim 1,
wherein the processor is configured to:
divide each of the plurality of reference documents into a plurality of partial documents;
determine whether or not each partial document overlaps with other partial documents; and
for a partial document determined as overlapping, display the partial document displayed later in a non-highlighted manner with respect to the partial document displayed earlier in accordance with a display order of the plurality of reference documents.

3. The document creation support apparatus according to claim 2,
wherein the processor is configured to exclude the partial document having a certain number of characters or less from an overlap determination target.

4. The document creation support apparatus according to claim 1,
wherein the processor is configured to, in a case where a ratio of an amount of the overlapping part to an amount of the reference document is less than or equal to a threshold value, not display the overlapping part included in the reference document in a non-highlighted manner with respect to the non-overlapping part.

5. The document creation support apparatus according to claim 1,
wherein the processor is configured to, in a case where a ratio of an amount of the overlapping part to an amount of the reference document is greater than or equal to a threshold value, not display at least a part of the overlapping part included in the reference document.

6. The document creation support apparatus according to claim 1,
wherein each of the plurality of reference documents is associated with a first degree of importance, and
the processor is configured to, in a case where the plurality of reference documents are displayed on the reference document display screen, display the reference document of which the first degree of importance is relatively high in a highlighted manner with respect to the reference document of which the first degree of importance is relatively low.

7. The document creation support apparatus according to claim 6,
wherein the processor is configured to display the overlapping part included in the reference document of which the first degree of importance is relatively high in a non-highlighted manner with respect to the non-overlapping part.

8. The document creation support apparatus according to claim 6,
wherein the processor is configured to not display the overlapping part included in the reference document of which the first degree of importance is relatively high in a non-highlighted manner with respect to the non-overlapping part.

9. The document creation support apparatus according to claim 2,
wherein each of the plurality of partial documents is associated with a second degree of importance, and
the processor is configured to, in a case where the plurality of reference documents are displayed on the reference document display screen, display the partial document of which the second degree of importance is relatively high in a highlighted manner with respect to the partial document of which the second degree of importance is relatively low.

10. The document creation support apparatus according to claim 9,
wherein the processor is configured to, for the partial document of which the second degree of importance is relatively high, display the partial document determined as overlapping in a non-highlighted manner with respect to the partial document determined as not overlapping.

11. The document creation support apparatus according to claim 9,
wherein the processor is configured to not display the partial document of which the second degree of importance is relatively high and that is determined as overlapping, in a non-highlighted manner with respect to the partial document determined as not overlapping.

12. The document creation support apparatus according to claim 1,
wherein the processor is configured to, in a case where a ratio of match between the reference documents is greater than or equal to a threshold value, determine that the reference document used for deriving the ratio of match includes the overlapping part.

13. The document creation support apparatus according to claim 12,
wherein the processor is configured to, in a case where the ratio of match between the reference documents is greater than or equal to the threshold value and a specific keyword or numerical value information is different between the reference documents, determine that the reference document used for deriving the ratio of match does not include the overlapping part.

14. The document creation support apparatus according to claim 12,
wherein the processor is configured to assign a weight to a specific keyword in deriving a ratio of match between the reference documents.

15. The document creation support apparatus according to claim 1,
wherein the processor is configured to, in a case where a plurality of the reference documents are displayed on the reference document display screen, not display at least a part of the reference document for which all partial documents included in the reference document overlap.

16. The document creation support apparatus according to claim 1,
wherein the processor is configured to:
receive selection of the reference document on the reference document display screen; and
copy at least a part of the selected reference document to the subject document creation screen.

17. The document creation support apparatus according to claim 16,
wherein the processor is configured to, in a case where the selected reference document includes the overlapping part, copy a part of the selected reference document excluding the overlapping part to the subject document creation screen.

18. The document creation support apparatus according to claim 16,
wherein the processor is configured to:
receive selection of a plurality of the reference documents on the reference document display screen; and
in a case where each of the plurality of selected reference documents includes the same overlapping part, copy a part of one of the selected reference documents other than the same overlapping part excluding the same overlapping part to the subject document creation screen.

19. The document creation support apparatus according to claim 1,
wherein the processor is configured to display the reference document including the same overlapping part in a state where a user is capable of performing an operation of tracking the same overlapping part.

20. The document creation support apparatus according to claim 1,
wherein the processor is configured to display a list of the reference documents including the same overlapping part.

21. The document creation support apparatus according to claim 2,
wherein the processor is configured to, in a case where the partial document determined as overlapping includes an examination item or a medical practice name, display, for the partial document, the partial document displayed later in order than a point in order corresponding to the number of executions of an examination indicated by the examination item or a medical practice indicated by the medical practice name in a non-highlighted manner with respect to the partial document displayed earlier in order in accordance with the display order.

22. The document creation support apparatus according to claim 2,
wherein the processor is configured to:
in a case where the partial document determined as overlapping includes an examination item or a medical practice name, divide a period designated as a period as a target for creating the subject document into a plurality of periods by separating the period on an execution date of an examination indicated by the examination item or a medical practice indicated by the medical practice name; and
for the partial document that is determined as overlapping and that includes the examination item or the medical practice name in each period, display the partial document displayed later in a non-highlighted manner with respect to the partial document displayed earlier in accordance with the display order of the plurality of reference documents.

23. The document creation support apparatus according to claim 2,
wherein the processor is configured to, for the partial document determined as overlapping, vary a display aspect of the partial document in accordance with the number of overlapping partial documents.

24. A document creation support method comprising:
via a processor included in a document creation support apparatus,
displaying a subject document creation screen for creating a subject document and a reference document display screen for displaying a reference document to be used for creating the subject document; and
displaying, in a case where a plurality of the reference documents are displayed on the reference document display screen, an overlapping part included in the reference document in a non-highlighted manner with respect to a non-overlapping part.

25. A non-transitory computer-readable storage medium storing a document creation support program for causing a processor included in a document creation support apparatus to execute a process comprising:
displaying a subject document creation screen for creating a subject document and a reference document display screen for displaying a reference document to be used for creating the subject document; and
displaying, in a case where a plurality of the reference documents are displayed on the reference document display screen, an overlapping part included in the reference document in a non-highlighted manner with respect to a non-overlapping part.
